(19) United States
(12) Patent Application Publication
(10) Pub. No.: US 2013/0071856 A1
(43) Pub. Date: Mar. 21, 2013

(54) METHOD AND BIOMARKER FOR EVALUATING METASTASIS, AND SIRNA COMPOUND FOR INHIBITING METASTASIS

(71) Applicants: Pao-Chi LIAO, Tainan City (TW); Hung-Chi Cheng, Tainan City (TW); Ying-Hwa Chang, Taipei City (TW); Shu-Hui Lee, Changhua County (TW)

(72) Inventors: Pao-Chi LIAO, Tainan City (TW); Hung-Chi Cheng, Tainan City (TW); Ying-Hwa Chang, Taipei City (TW); Shu-Hui Lee, Changhua County (TW)

(21) Appl. No.: 13/600,713

(22) Filed: Aug. 31, 2012

Related U.S. Application Data
(60) Provisional application No. 61/530,003, filed on Sep. 1, 2011.

(51) Int. Cl. G01N 33/68

(52) U.S. Cl. CPC G01N 33/6893
USPC 435/7.8; 536/23.1; 530/350; 530/389.1; 536/24.5

(57) ABSTRACT

A method and biomarker for evaluating metastasis, and an siRNA compound for inhibiting metastasis. The method of the present invention includes: providing a sample of a subject, which includes a normal tissue and a tissue to be detected; detecting expression of a biomarker of the normal tissue and the tissue to be detected, respectively, wherein the biological marker is SERPINA1; and comparing the expression of the biological marker of the normal tissue and the tissue to be detected. When the expression of the biological marker of the tissue to be detected is higher than the normal tissue, it represents that the subject is at a risk of suffering from metastasis.

### CROSS REFERENCE TO RELATED APPLICATION

This application claims the benefit of filing date of U.S. Provisional Application Ser. No. 61/530,003, entitled “FN1, TIMP1, and SERPINA1 Regulate the Migration, Invasion, and Pericellular Fibronectin Assembly of Cancer Cells in Metastasis: A Novel Biological Significance Revealed by Quantitative Proteomics Analysis of NSCLC Cell Secretomes” filed Sep. 1, 2012 under 35 USC §119(e)(1).

### BACKGROUND OF THE INVENTION

1. Field of the Invention

The present invention relates to a method and a biomarker for evaluating metastasis, and an siRNA compound for inhibiting metastasis. More specifically, the present invention relates to method and a biomarker for evaluating an incidence rate of metastasis, and siRNA compound for inhibiting metastasis derived therefrom.

2. Description of Related Art

Lung cancer is a major factor causing death around the world, and 90% lung cancer patients died due to metastasis. Metastasis is the spread tumor cells from lung to other organs. Once the metastasis occurs, the symptoms of cancers are hardly to control and cure. In addition, metastasis involves in complicated mechanisms, wherein the tumor cells with invasive or migration capacities separate from primary organs, invade to peripheral blood vessel or lymph capillary, and spread to other non-adjacent organs via blood circulation or lymphatic system to form a secondary cancer.

In addition, there are several types of lung cancer including small cell lung cancer (SCLC) and non-small cell lung cancer (NSCLC). Recently, most of lung cancer patients are suffered from NSCLC. NSCLC is divided into three sub-types including adenocarcinoma, squamous cell lung carcinoma and large cell lung carcinoma. 40% of lung cancers are adenocarcinoma, 30% thereof are squamous cell lung carcinoma, and 9% of large cell carcinoma.

Some studies indicate that tumor cells have to interact with specific molecules or factors in secretome during metastasis, and some of the specific molecules or factors are regulated by secretory factors with catalytic or recognition properties. Hence, it may be helpful to prevent and diagnose metastasis if the regulation related to secretome can be analyzed, the relations between each proteins and cancers can be identified by several experimental manners, important proteins highly related to metastasis can be selected from the secretome, and the relation between the selected proteins and cancers can be well-understood.

Since secretome is highly related to cancer metastasis, it is desirable to identify proteins related to cancer metastasis through an effective method for analyzing secretome, and then the identified proteins can be used to evaluate metastasis or develop drugs for inhibiting metastasis before secondary cancers formed or at early stages of metastases. Hence, the metastasis can be prevented or found in early stages, so the incidence rate of metastasis can be reduced and the mortality from cancers can further be decreased.

### SUMMARY OF THE INVENTION

An object of the present invention is to provide a method for evaluating metastasis, which can be used to evaluate whether a subject is at a risk of suffering from metastasis.

Another object of the present invention is to provide a biomarker for evaluating metastasis, which can be used to evaluate an incidence rate of metastasis in a subject. In addition, the biomarker for evaluating metastasis can further be used as a target in a gene therapy or used in a protein drug development.

A further object of the present invention is to provide an siRNA compound for inhibiting lung metastasis, which can be used in a gene therapy of lung cancer treatment. To achieve the object, the method for evaluating metastasis of the present invention comprises the following steps: (A) providing a sample of a subject including a normal tissue and a tissue to be detected; (B) detecting expression of a biomarker in the normal tissue and the tissue to be detected respectively, wherein the biomarker is serpin peptidase inhibitor, clade A α1-antitrypsin (SERPINA1); and (C) comparing the expression of the biomarker in the normal tissue with that in the tissue to be detected, wherein when the expression of the biomarker in the normal tissue is higher than that in the tissue to be detected, this indicates that the subject is at a risk of suffering from metastasis. Herein, the normal tissue can be a normal organ tissue or a normal blood tissue, and the tissue to be detected can be a tumor tissue or a blood tissue from a subject with lung cancer.

In addition, the biomarker for evaluating metastasis, which is at least one selected from a group consisting of a nucleotide sequence, a complementary sequence of the nucleotide sequence, a derivative of the nucleotide sequence, an amino-acid sequence, a derivative of the amino-acid sequence, a fragment of the amino-acid sequence, a mutation of the amino-acid sequence, and an antibody corresponding to the amino-acid sequence of SERPINA1. Preferably, the biomarker is at least one selected from the group consisting of the amino-acid sequence, the derivative of the amino-acid sequence, the fragment of the amino-acid sequence, the mutation of the amino-acid sequence, and the antibody corresponding to the amino-acid sequence of SERPINA1.

Although it is known that SERPINA1 is a type of serine protease inhibitor, the role of SERPINA1 is still unidentified. Some studies indicate that the expression of SERPINA1 in serum of cancer patients is much higher than that in health people. The present inventors found that SERPINA1 participates in several cancer metastases, including ovarian cancer, cervical cancer, colorectal cancer, breast cancer and lung cancer (especially, non-small lung cancer (NSCLC)). In addition, the present inventors also found that SERPINA1 participates in regulation of the invasive and migration capacities of lung tumor cells such as CL1-5, and regulates the aggregation of fibronectin (FN1) on surfaces of tumor cells, wherein the aggregation of FN1 may increase the probability of metastasis. Therefore, the present invention uses SERPINA1 as a biomarker for evaluating metastasis, based on the aforementioned foundings.

In the method for evaluating metastasis, the lung cancer can be small cell lung cancer (SCLC) or non-small cell lung cancer (NSCLC). Preferably, the lung cancer is NSCLC.

In addition, the biomarker used in the present invention may have a nucleotide sequence with 50% or more identity to a sequence represented by SEQ ID NO: 1. Preferably, the biomarker of the present invention has nucleotide sequence with 70-100% identity to a sequence represented by SEQ ID NO: 1. More preferably, the biomarker of the present invention has nucleotide sequence with 90-100% identity to a sequence represented by SEQ ID NO: 1. In addition, the biomarker used in the present invention may have an amino-acid sequence with 50% or more identity to a sequence represented by SEQ ID NO: 2. Preferably, the biomarker of the present invention has amino-acid sequence with 70-100% identity to a sequence represented by SEQ ID NO: 2. More preferably, the biomarker of the present invention has amino-acid sequence with 90-100% identity to a sequence represented by SEQ ID NO: 2. In the present invention, the term “identity” refers to the percentage of identical components, i.e. the percentage of identical nucleotides or amino-acid residues in the nucleotide sequence or the amino-acid sequence.

The step (B) of the method for evaluating metastasis of the present invention may be: detecting the expression of mRNA, protein, protein derivatives, or protein fragments of SERPINA1 in the normal tissue or the tissue to be detected respectively. Preferably, the step (B) is: detecting the expression of protein of SERPINA1 in the normal tissue or the tissue to be detected respectively.

Any techniques generally known in the art can be used in the method for evaluating metastasis of the present invention to identify the expression of SERPINA1 in the normal tissue or the tissue to be detected, for example, western blot analysis, electrophoresis, enzyme-linked immunosorbent assay (ELISA), immunohistochemistry (IHC), immunoprecipitation (IP), mass spectrometry (MS), real time polymerase chain reaction (RT-PCR) or real time quantitative polymerase chain reaction (real time Q-PCR). Preferably, the expression of protein of SERPINA1 in the normal tissue or the tissue to be detected are detected respectively through western blot analysis, electrophoresis, enzyme-linked immunosorbent assay (ELISA), immunohistochemistry (IHC), immunoprecipitation (IP) or mass spectrometry (MS).

Since secretome is highly related to metastasis, SERPINA1 in secretome has potential for evaluating metastasis. Hence, the method of present invention is performed by detecting the expression of SERPINA1 in a sample from a subject to evaluate the risk or the incidence rate of metastasis. Therefore, it is possible to find and exactly estimate the metastatic tumor cells before a secondary cancer is formed or at an early stage of the secondary cancer, so the survival rate of lung cancer patients can further be increased. Furthermore, the biomarker of the present invention may further be used in metastasis predictions or as prognostic indicators in clinic, and applied to targeting therapy.

Furthermore, the present invention also provides an siRNA compound for inhibiting lung metastasis, which comprises: a target sequence, which is selected from genes of SERPINA1. The siRNA compound of the present invention can be used in RNA interference (RNAi) gene therapy to inhibit the invasion and migration of lung cancer, so the effect of cancer treatment can be improved and the mortality from cancers can be decreased.

In the siRNA compound for inhibiting lung metastasis of the present invention, the target sequence may comprise 20-25 continuous nucleotides from SERPINA1. Preferably, the target sequence comprises 20-25 continuous nucleotides from SERPINA1 represented by SEQ ID NO: 1.

Other objects, advantages, and novel features of the invention will become more apparent from the following detailed description when taken in conjunction with the accompanying drawings.

### BRIEF DESCRIPTION OF THE DRAWINGS

FIG. 1 is a result of western blotting analysis of SERPINA1 of the present invention;

FIG. 2 is a result of wound-healing assay of the present invention;

FIG. 3 is a result of migration assay of the present invention;

FIG. 4 is a result of matrigel invasion assay of the present invention;

FIG. 5 is a result of flow cytometry of CL1-0 cells of the present invention;

FIG. 6 is a result of flow cytometry of CL1-5 cells of the present invention;

FIG. 7 is a result of flow cytometry of a control group of the present invention;

FIG. 8 is a result of flow cytometry of an experimental group of the present invention; and

FIG. 9 is a result of lung colony assay of the present invention.

### DETAILED DESCRIPTION OF THE PREFERRED EMBODIMENT

### Lung Cancer Cell Line CL1.

In the present embodiment, lung cancer cell lines (CL1-0 and CL1-5 cells) with different invasive and metastatic capabilities were provided by Department of Internal Medicine, National Taiwan University Hospital, Taipei, Taiwan, Republic of China. The cells were maintained in an RPMI 1640 medium supplemented with 10% fetal bovine serum (FBS) and antibiotics at 37° C. under 5% CO₂.

Harvest of Conditioned Media from Lung Cancer Cell Lines

CL1 cells were grown to confluence in tissue culture dishes, washed with serum-free media three times to avoid serum contamination, and incubated in serum-free media for 24 h. The supernatants of the conditioned media (CM) were then harvested and centrifuged to eliminate the intact cells and contaminants. Next, the supernatants were concentrated and desalted by centrifugation in Amicon Ultra-15 tubes (molecular weight cutoff 3000 Da; Millipore, Billerica, Mass.). The protein concentrations of CL1 CM samples were determined using the Bradford protein assay reagent (Biorad). Herein, the protein concentrations obtained by Bradford protein assay reagent were the concentrations of secretome.

### Separation and Purification of Secretome

The obtained secretome from concentrated CM samples was purified with a purification gel. Herein, the purification gel was prepared as follows. First, 0.6 mL of H₂O, 2.22 mL of 1.5 M Tris-HCl [pH 8.8], 90 μL of 10% SDS, 6 mL of Bis/Acrylamide, 90 μL of 10% ammonium persulfate, and 5 μL of TEMED were mixed well and set to polymerize for 1 hour to obtain a resolving gel portion (i.e. high-density layer). Then, 2.9 mL of H₂O, 0.5 mL of 1 M Tris-HCl [pH 6.8], 40 μL of 10% SDS, 520 μL of Bis/Acrylamide, 40 μL of 10% ammonium persulfate, and 4 μL of TEMED were mixed and poured on the resolving gel portion. After a setting process was completed, a stacking gel portion (i.e. low-density layer) was obtained. After the aforementioned process, a purification gel was obtained, which comprises a resolving gel portion (i.e. high-density layer) and a stacking gel portion (i.e. low-density layer). In addition, the low-density layer was stacked on the high-density layer.

A total of 100 μg of secretome was mixed with 13 μL of H₂O, 5 μL of 4 × SDS sample buffer, and 2 μL of 0.5M DTT and then boiled under 95° C. for 10 min. The purification was run at 55 V. The electrophoresis was stopped after the sample had just passed into the resolving gel portion, and the gels were then stained using Coomassie Brilliant Blue (CBB) R-250.

### In-Gel Digestion

The secretome sample located on an interface between the low-density layer and the high-density layer was collected and the gel pieces were diced into about 1 mm³. Gel slices were washed and dehydrated three times in 25 mM ammonium bicarbonate (ABC) (pH 7.9) and 50 mM ABC/50% acetonitrile. A protein reduction was subsequently performed by incubating 0.5M DTT for 1 h at 56° C. and then alkylating with 50 μL saturated IAA for 45 min at room temperature in the dark (i.e. carbamidomethylation process). After two subsequent wash/dehydration cycles, each gel sample was digested with 4 μg (1:25, w/w) of sequencing-grade modified trypsin (Promega)/25 mM ammonium bicarbonate and incubated at 37° C. for an overnight digestion (16-18 hours). After the digestion process, peptides, which were obtained from the secretome, were extracted twice in 100 μL of 50% ACN in 5% formic acid. The extracted peptides were enriched using OMIX C18 pipet tips (Varian) to remove any contaminants, which may have affected the signal of the sequential iTRAQ labeling.

Isotope Labeling of Peptides from Secretome

The enriched peptides from the secretome were labeled with the iTRAQ reagent (Applied Biosystems, Foster City, Calif., USA) according to the manufacturer's protocol.

Briefly, one unit of iTRAQ reagent was thawed and reconstituted in ethanol (70 μL), wherein one unit was defined as the amount of reagent required to record 100 μg of protein. The obtained peptide mixtures were reconstituted with 20 μL of iTRAQ dissolution buffer. 70 μL iTRAQ reagent solutions (iTRAQ115:iTRAQ116=1:1, or iTRAQ114:iTRAQ117=1:1) were combined with the peptide mixtures from the secretomes. The extracted peptide mixtures were then pooled and dried by vacuum centrifugation. The dried peptide mixture was reconstituted and acidified with 10 μL of buffer (5 mM K₂HPO₄ and 25% ACN [pH 3]) for fractionation by SCX chromatography using an AKTA FPLC system (GE Healthcare) to reduce the complication of the samples. A total of 28 fractionations were generated and were desalted using OMIX C18 pipet tips (Varian) according to the user instructions in order to remove the salts which may influence the signal of isotope reagents.

Analysis of Peptides from Secretome with LC-ESI-MS/MS

iTRAQ-labeled samples were reconstituted in eluent buffer A (0.1% (v/v) FA in H2O) and analyzed by LCMS/MS. The buffer B (0.1% (v/v) FA in ACN) gradient started from 0% to 5% at 2 mins and then progressed to 37% in 140 mins. Peptides were eluted at 200-300 nL/min.

Peptide fragmentation by collision-induced dissociation was performed automatically using the information-dependent acquisition in Analyst QS v1.1 (Applied Biosystems). The method applied a 1-s TOF MS scan and automatically switched to three 2-s product ion scans (MS/MS) when a target ion reached an intensity of greater than 20 counts. TOF MS scanning was undertaken over the range 400-2000 m/z. Product ion scans were undertaken over the range 100-2000 m/z at low resolution.

### Database Comparison

The results from LC-MS/MS were batch-searched against the Swiss-Prot human sequence database (version 20090616; 468851 sequences) using the MASCOT algorithm (v2.1.0, Matrix Science, London, U.K.). The peak list in the MS/MS spectra generated under ESI-Q-TOF was extracted with AnalystQS 1.1 (Applied Biosystems) with the default charge state set to 2+, 3+, and 4+. The MS and MS/MS centroid parameters were set to 10% height percentage and to a merge distance of 0.1 amu. For the MS/MS grouping, the averaging parameters consisted of rejection of spectra with less than five peaks or precursor ions with less than 10 counts/s. Search parameters for peptide and for MS/MS mass tolerance were 1 and 0.5 Da, respectively, with allowance for two missed cleavages made in the trypsin digest and for variable modifications of deamidation (Asn, Gln), oxidation (Met), iTRAQ (Nterminal), iTRAQ (Lys), and carboxyamidomethylation (Cys). Peptides were considered to have been identified if their MASCOT individual ion score was higher than the MASCOT score 20.

After the aforementioned analysis, 331 proteins were identified from the secretome of lung cancer samples.

### Protein Quantification

For protein quantification, data analysis for the iTRAQ experiments was performed with the software Multi-Q. The raw data files from QSTAR Pulsar I were converted into files of mzXML format by the program mzFAST, and the search results in MASCOT were exported in comma-separated-values (CSV) data format. After the data conversions, Multi-Q selected iTRAQ labeled peptides with confident MS/MS identifications (MASCOT score 20), detected signature ions (m/z 114, 115, 116, and 117), and performed an automated quantification of peptide abundance.

To calculate the average protein ratios, the ratios of quantified, unique iTRAQ peptides were weighted according to their peak intensities to minimize standard deviation.

### Bioinformatics Analysis

The identified proteins were analyzed using the SignalP, SecretomeP, and TMHMM programs to predict the possibility of protein secretion through classic or through nonclassic secretion pathways and the presence of transmembrane domains in the protein sequence. The molecular functions of the identified proteins were determined based on a search against the Human Protein Reference Database (HPRD) (http://www.hprd.org/).

After the bioinformatics analysis, more than 77.3% of identified proteins may be assumed to be secreted proteins through different secretion pathways. In addition, 66 proteins were identified through the bioinformatics analysis, which have significant expression differences in CL1-0 and CL1-5 and may be related to lung metastasis.

### Statistical Analysis

All experiments were performed in triplicate, and the results are shown as the mean±SD. The nonparametric Mann-Whitney U test was employed to analyze the comparison between two groups. P values less than 0.05 were considered statistically significant.

### Western Blotting Analysis

12 proteins are selected from the identified 66 proteins related to lung metastasis, which includes Nidogen-1, MAGE-A4, PRDX1, CKB, PLAU, SERPINA1,

TIMP, FN1, HSPA5, COL6A1, THBS1, and CTSL1. These 12 proteins were examined through western blotting analysis, in order to identify whether these proteins were indeed related to lung metastasis. First, 5-30 μg of secreted proteins from the CL1 cell CMs were separated on a 12% SDS-PAGE and transferred to PVDF membranes (Millipore). The membranes were blocked in a 5% nonfat milk solution for 1 hour at room temperature and then probed with various antibodies against the selected proteins (Santa Cruz Biotechnology) and against anti-α-tubulin (Calbiochem) for 3 hours. The membranes were washed with TBST 3 times and incubated with horseradish peroxidase-conjugated secondary antibodies at a dilution of 1:5000 at room temperature for 1 hr. The membranes were washed with TBST 5 times before developing them with enhanced chemiluminescence detection.

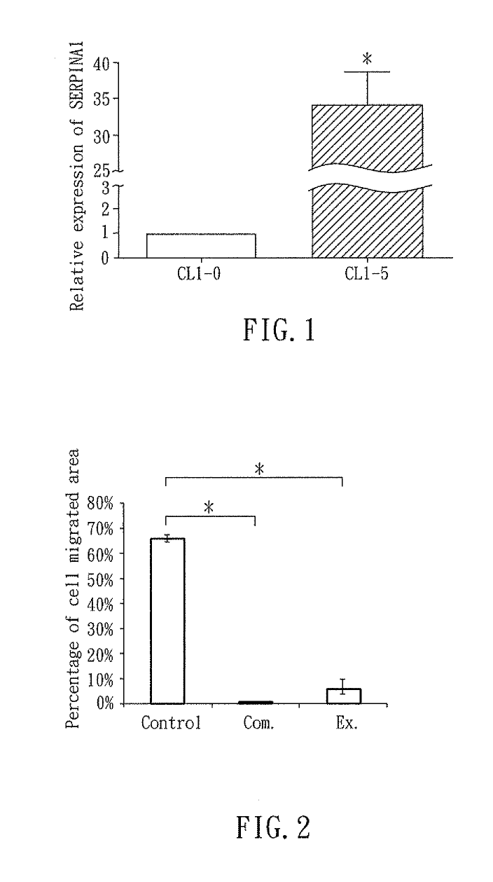

The results of western blotting analysis show that the expression of PLAU, SERPINA1, TIMP, FN1, HSPA5, COL6A1, THBS1 and CTSL1 can be identified in the CL1-5 with high invasive capacity, and the expression of Nidogen-1, MAGE-A4, PRDX1 and CKB can be identified in the CL1-0 with low invasive capacity. In addition, FIG. 1 shows the experimental result about SERPINA1. As shown in FIG. 1, the expression of SERPINA1 in CL1-5 is much higher than that in CL1-0, and shows significant differences. Hence, according to the results of western blotting analysis, the proteins highly related to lung metastasis, especially SERPINA1 of the present invention can be identified through the aforementioned gel purification, isotope labeling, and mass spectrometry of the present embodiment.

siRNA Interference

There are no studies showing that SERPINA1 is related to metastasis. Herein, SERPINA1 gene silencing was performed to identify the relation between SERPINA1 and metastasis.

In the present analysis, SERPINA1 siRNA was provided, which was a mixture containing three DNA sequence sets represented by a set consisting of SEQ ID NO: 3 and SEQ ID NO: 4, a set consisting of SEQ ID NO: 5 and SEQ ID NO: 6, and a set consisting of SEQ ID NO: 7 and SEQ ID NO: 8. In addition, FN1 siRNA was also provided, which was a mixture containing three DNA sequence sets represented by a set consisting of SEQ ID NO: 9 and SEQ ID NO: 10, a set consisting of SEQ ID NO: 11 and SEQ ID NO: 12, and a set consisting of SEQ ID NO: 13 and SEQ ID NO: 14. Then, CL1-5 cells were transfected with the aforementioned siRNAs using the siRNA transfection reagent according to the manufacturer's instructions (Santa Cruz Biotechnology, Santa Cruz, Calif.). For each transfection, 80 pmol of the SERPINA1 siRNA, FN1 siRNA or control siRNA (scramble siRNA) with 6 μL of siRNA transfection reagent was added to 100 μL of siRNA transfection media. The solution was mixed gently and overlaid onto the CL1-5 cells for 24 h. The media was then aspirated and 3×10⁵ CL1-5 cells were grown in 2 mL of RPMI 1640 containing 10% fetal bovine serum (FBS) on six-well culture dishes reaching 80% confluence at 37° C. under 5% CO₂. Herein, the experimental group (Ex.) was CL1-5 cells transfected with SERPINA1 siRNA, the control group (Control) was CL1-5 cells transfected with scramble siRNA, and the comparative group (Comp.) was CL1-5 cells transfected with FN1 siRNA. Then, the aforementioned western blotting analysis was performed to identify the results of siRNA interference. In addition, the CL1-5 cells transfected with the aforementioned siRNA were further used to perform the following wound healing assay, migration assay and matrigel invasion assay.

The results of siRNA interference show that the transfection of SERPINA1 siRNA can inhibit the protein expression of SERPINA1 in CL1-5 cells, and the transfection of FN1 siRNA also can inhibit the protein expression of FN1 in CL1-5 cells. Hence, the SERPINA1 siRNAs used in the present embodiment has effect on inhibiting the expression of SERPINA1 protein.

### Wound-Healing Assay

Cell migration ability was examined with the commercial ibidi Culture-Insert (Applied BioPhysics, Inc., Troy, N.Y., USA). Cells were seeded on the insert for 12 hrs, and the inserts were removed. Photographs were taken at 0 hr and 24 hrs at the same position in the cell-free gap insert with 100× magnification. The Image-Pro Plus 6.0 software was used to calculate the cell migrating area (Media Cybernetics, Inc. Bethesda, Md., USA).

The results show that SERPINA1 siRNA led to a dramatic decrease of invasion in the SERPINA1-siRNA-transfected CL1-5 cells in comparison with the scramble-siRNA-transfected CL1-5 cells. These results demonstrate that the knock-down of SERPINA1 expression impairs migration and invasion in CL1-5 cells and that SERPINA1 is critical for migration and invasion in CL1-5 cells. In addition, FN1 siRNA also led to a dramatic decrease of invasion in the FN1-siRNA-transfected CL1-5 cells, as shown in FIG. 2.

### Migration Assay

A transwell membrane (8-μm pore size, BD Biosciences) was used for a transwell migration assay, The CL1 cells were trypsinized, washed, and kept suspended in their medium without FBS. To the lower wells of the chambers, a migration inducing medium (with 10% FBS) was added. The upper wells were filled with a serum-free medium with cells (100,000 cells per well), and the lower chambers were filled with an RPMI 1640 medium supplemented with 10% FBS to induce cell migration. After 24 hours, the assays were stopped by the removal of the medium from the upper wells and the careful removal of the filters. The filters were fixed with methanol and then stained with 20% Giemsa solution (Sigma). The cell number on each filter was counted under a microscope (200×), and 6 fields were randomly selected on each filter for further statistical analysis.

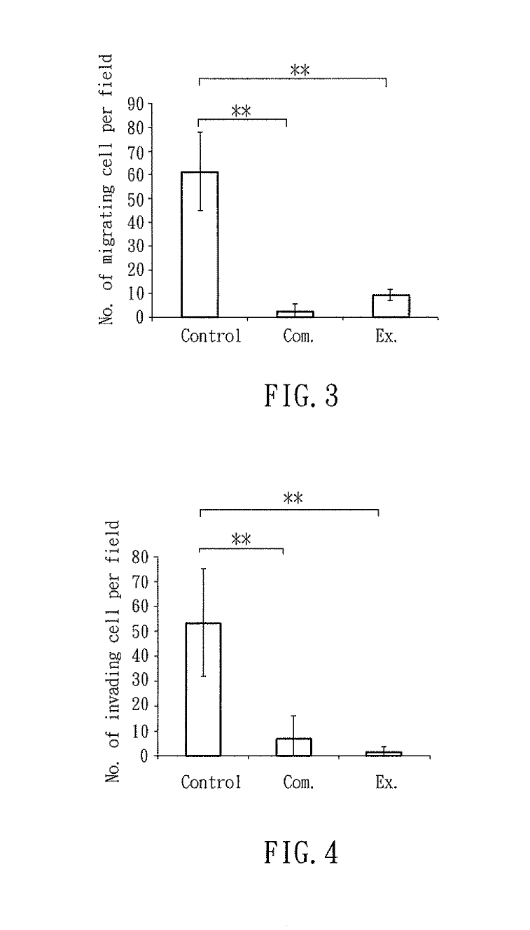

As shown in FIG. 3, a decrease in migration was observed in the SERPINA1-siRNA-transfected CL1-5 cells (experimental group, Ex.) in comparison with the scramble-siRNA transfected CL1-5 cells (control group, Control) due to RNA interferencing. In addition, a decrease in migration was observed in the FNI-siRNA-transfected CL1-5 cells (comparative group, Comp.).

### Matrigel Invasion Assay

Cell invasion was examined in a membrane invasion culture system. A transwell membrane (8-μm pore size, BD Biosciences) coated with Matrigel basement membrane matrix (2.5 mg/mL; BD Biosciences Discovery Labware) was used for the invasion assay. Cells (1×10⁵) were seeded into the upper wells in an RPMI 1640 medium, and the lower chambers were filled with an RPMI 1640 medium supplemented with 10% FBS. After incubating at 37° C. for 24 h, the membranes were fixed with methanol and the cells were stained with Giemsa staining. The cell number on each filter was counted under a microscope (200×).

The results show that a decrease in invasion was observed in the SERPINA1-siRNA-transfected CL1-5 cells (experimental group, Ex.) in comparison with the scramble-siRNA transfected CL1-5 cells (control group, Control) due to RNA interferencing. In addition, a decrease in invasion was observed in the FN1-siRNA-transfected CL1-5 cells (comparative group, Comp.), as shown in FIG. 4.

### Flow Cytometry

Fluorescence-activated cell sorting (FACS) was performed to quantify FNI expression on the cell surfaces. The CL1-0 and CL1-5 cells were trypsinized and incubated in suspension for 2 hrs in 20% FBS media and were washed once with PBS. Cells were incubated with a rabbit anti-FNI antibody (diluted 1:600 in PBS with 1% BSA, Sigma) for 1 hr at 4° C. before they were stained with a fluorescein isothiocyanate-conjugated donkey anti-rabbit antibody in PBS containing 1% BSA for 1 hr at 4° C. and fixed in 2% paraformaldehyde in PBS. FACS analysis was performed on a Coulter Epics Profile (FACSCalibur, BD Biosciences, San Jose, Calif., USA). The nonspecific fluorescence was accounted for by incubating the tumor cells with non-immune serum rather than the primary antibody.

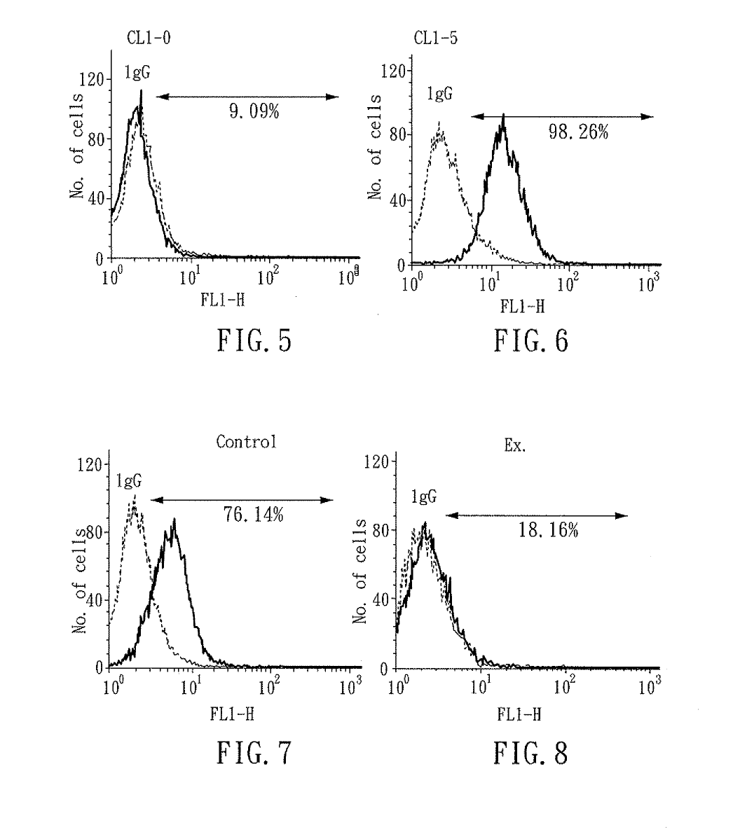

As shown in FIG. 5, the expression of FN1 can be found on surfaces of only 9.09% CL1-0 cells with low invasive capacity. As shown in FIG. 6, the expression of FN1 can be found on surfaces of 98.26% CL1-5 cells with high invasive capacity. As shown in FIG. 7, the expression of FN1 can be found on surface of 76.16% scramble-siRNA-transfected CL1-5 cells. However, as shown in FIG. 8, the expression of FN1 can be found on surface of only 18.16% SERPINAl-siRNA-transfected CL1-5 cells, and it is because that the expression of SERPINA1 was knockout by SERPINA1 siRNA. These results indicate that SERPINA1 may regulate FN1 aggregating on cell surfaces to inhibit cancer metastasis.

### Lung Colony Assay

The transfected cells were incubated in suspension for 2 hrs in 20% FBS media. Eight-week-old nude mice were injected in the lateral tail vein with a single-cell suspension that contained 2×10⁶ cells in 0.2 mL RPM1-1640 base medium. The mice were sacrificed after 8 weeks, and the lungs were removed and fixed in 3.7% formalin fixative. The representative lung tumors were removed, fixed, and immediately embedded in paraffin, which was sectioned into 4-mm layers and stained with hematoxylin and eosin (H&E) for histologic analysis.

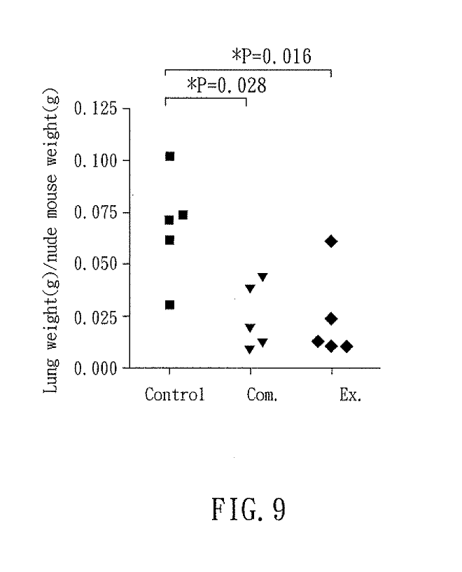

The result of the present experiment is shown in a ratio of lung weight to nude mouse weight. The metastatic tumor cells in lung and the lung weight of the mice treated with SERPINA1-siRNA-transfected CL1-5 cells were fewer and lighter than those treated with scramble-siRNA-transfected CL1-5 cells, due to the siRNA of SERPINA1. In addition, fewer metastatic tumor cells in lung were also found in the mice treated with FN1-siRNA-transfected CL1-5 cells, as shown in FIG. 9. Furthermore, H&E staining of mouse lungs confirmed that the pulmonary alveoli were filled with metastatic tumor cells in the mice treated with scramble-siRNA-transfected CL 1-5 cells. However, there was a sufficient space in pulmonary alveoli of the mice treated with SERPINA1-siRNA-transfected CL1-5 cells, and only few pulmonary alveoli were filled with metastatic tumor cells. This is because the SERPINA1 siRNA can interfere in the expression of SERPINA1. In addition, there was also a sufficient space in pulmonary alveoli of the mice treated with FN1-siRNA-transfected CL1-5 cells.

In conclusion, both the migration and invasive capacity of CL1-5 lung tumor cells indeed can be reduced by inhibiting the expression of SERPINA1 protein. In addition, the aggregation of FN1 protein on surfaces of tumor cells can also be reduced by inhibiting the expression of SERPINA1 protein. The present invention confirms that SERPINA1 protein is highly related to the invasion/migration of lung tumor cells. Hence, when the RNAi gene therapy of the present invention is applied, the expression of SERPINA1 can be reduced, and therefore the cancer metastasis can further be inhibited.

Although the present invention has been explained in relation to its preferred embodiment, it is to be understood that many other possible modifications and variations can be made without departing from the spirit and scope of the invention as hereinafter claimed.

What is claimed is:

1. A method for evaluating cancer metastasis, comprising following steps:
(A) providing a sample of a subject including a normal tissue and a tissue to be detected;
(B) detecting expression of a biomarker in the normal tissue and the tissue to be detected respectively, wherein the biomarker is SERPINA1; and
(C) comparing the expression of the biomarker in the normal tissue with that in the tissue to be detected, wherein when the expression of the biomarker in the normal tissue is higher than that in the tissue to be detected, this indicates that the subject is at a risk of suffering from metastasis.

2. The method as claimed in claim 1, wherein the normal tissue is a normal organ tissue or a normal blood tissue, and the tissue to be detected is a tumor tissue or a blood tissue from a subject with lung cancer.

3. The method as claimed in claim 1, wherein the expression of mRNA, protein, protein derivatives, or protein fragments of SERPINA1 in the normal tissue or the tissue to be detected are detected respectively in the step (B).

4. The method as claimed in claim 3, wherein the expression of protein of SERPINA1 in the normal tissue or the tissue to be detected are detected respectively.

5. The method as claimed in claim 4, wherein the expression of protein of SERPINA1 in the normal tissue or the tissue to be detected are detected respectively through western blot analysis, electrophoresis, enzyme-linked immunosorbent assay (ELISA), immunohistochemistry (IHC), immunoprecipitation (IP) or mass spectrometry (MS).

6. The method as claimed in claim 1, wherein a nucleotide sequence of SERPINA1 has 50% or more identity to a sequence represented by SEQ ID NO: 1.

7. The method as claimed in claim 1, wherein an amino-acid sequence of SERPINA1 has 50% or more identity to a sequence represented by SEQ ID NO: 2.

8. The method as claimed in claim 2, wherein the lung cancer is a non-small lung cancer.

9. A biomarker for evaluating metastasis, which is at least one selected from a group consisting of a nucleotide sequence, a complementary sequence of the nucleotide sequence, a derivative of the nucleotide sequence, an amino-acid sequence, a derivative of the amino-acid sequence, a fragment of the amino-acid sequence, a mutation of the amino-acid sequence, and an antibody corresponding to the amino-acid sequence of SERPINA1.

10. The biomarker as claimed in claim 9, which is at least one selected from the group consisting of the amino-acid sequence, the derivative of the amino-acid sequence, the fragment of the amino-acid sequence, the mutation of the amino-acid sequence, and the antibody corresponding to the amino-acid sequence of SERPINA1

11. The biomarker as claimed in claim 9, wherein the nucleotide sequence of SERPINA1 has 50% or more identity to a sequence represented by SEQ ID NO: 1.

12. The biomarker as claimed in claim 9, wherein the amino-acid sequence of SERPINA1 has 50% or more identity to a sequence represented by SEQ ID NO: 2. of SERPINA1 has 50% or more identity to a sequence represented by SEQ ID NO: 2.

13. An siRNA compound for inhibiting lung metastasis, comprising:
a target sequence, which is selected from genes of SERPINA1.

14. The siRNA compound as claimed in claim 13, wherein the target sequence comprises 20-25 continuous nucleotides from SERPINA1.

15. The siRNA compound as claimed in claim 13, wherein the nucleotide sequence of SERPINA1 is represented by SEQ ID NO: 1.


SEQUENCE LISTING (14 sequences)

SEQ ID NO: 1 - DNA, 13946 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggtgggagggacagcagggcttagagtgggggtcattgtgcagatgggaaaacaaaggcccagagaggggaagaaatgcccaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcgggcgatgcaatctgatttaggcttttaaagggattgcaatcaagtggggccccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 2 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - DNA, 21 bp, Artificial
  synthesized
ccaacagcaccaauaucuutt

SEQ ID NO: 4 - DNA, 21 bp, Artificial
  synthesized
aagauauuggugcuguuggtt

SEQ ID NO: 5 - DNA, 21 bp, Artificial
  synthesized
guccauuacuggaaccuautt

SEQ ID NO: 6 - DNA, 21 bp, Artificial
  synthesized
auagguuccaguaauggactt

SEQ ID NO: 7 - DNA, 21 bp, Artificial
  synthesized
cgaggucaaguucaacaaatt

SEQ ID NO: 8 - DNA, 21 bp, Artificial
  synthesized
uuuguugaacuugaccucgtt

SEQ ID NO: 9 - DNA, 21 bp, Artificial
  synthesized
gacuggugguuacauguuatt

SEQ ID NO: 10 - DNA, 21 bp, Artificial
  synthesized
uaacauguaaccaccaguctt

SEQ ID NO: 11 - DNA, 21 bp, Artificial
  synthesized
cgcaucacuugcacuucuatt

SEQ ID NO: 12 - DNA, 21 bp, Artificial
  synthesized
uagaagugcaagugaugcgtt

SEQ ID NO: 13 - DNA, 21 bp, Artificial
  synthesized
gauccugucuacuucacaatt

SEQ ID NO: 14 - DNA, 21 bp, Artificial
  synthesized
uugugaaguagacaggauctt